(19) United States
(12) Patent Application Publication
(10) Pub. No.: US 2023/0000605 A1
(43) Pub. Date: Jan. 5, 2023

(54) Midline Marker For Sling Implant

(71) Applicant: Caldera Medical, Inc., Agoura Hills, CA (US)

(72) Inventors: Jeffrey Wells, Agoura Hills, CA (US); Vishtasb Farkhondeh, Agoura Hills, CA (US); Sandra Muhlfeld, Agoura Hills, CA (US)

(73) Assignee: Caldera Medical, Inc., Agoura Hills, CA (US)

(21) Appl. No.: 17/810,241

(22) Filed: Jun. 30, 2022

Related U.S. Application Data
(60) Provisional application No. 63/217,447, filed on Jul. 1, 2021.

(51) Int. Cl. A61F 2/00

(52) U.S. Cl. CPC A61F 2/0045; A61F 2250/0091; A61F 2250/0097

(57) ABSTRACT

A sling assembly for treatment of stress urinary incontinence may comprise a midline marker, a sling, and a sleeve. The midline marker identifies the midpoint of the protective sleeve when it is installed on a transparent surface of the sleeve. The protective sleeve comprises a clear transparent surface and a colored opaque surface. During a procedure, the colored opaque surface of the sleeve provides contrast between the surfaces of the sleeve to identify any twisting present throughout the length of the sleeve. The clear transparent surface of the sleeve forms a channel at the center of the midline marker when the transparent surface of the sleeve is wrapped over the body of the midline marker. The channel is large enough for a surgical instrument to be inserted and cut the transparent surface to separate the sleeve from the midline marker after installation of the sling at a desired location.

### RELATED APPLICATIONS

This application claims priority to U.S. Provisional Application Ser. No. 63/217,447 filed Jul. 1, 2021 entitled Midline Marker for Sling Implant, which is hereby incorporated herein by reference in its entirety.

### BACKGROUND OF THE INVENTION

Anatomical tissues may become weakened or damaged by age, injury, or disease. The weakening of the structural integrity of anatomical tissues may have significant medical consequences. One such consequence is the development of Stress Urinary Incontinence (SUI) affecting primarily women, but also men. The most common treatment of SUI involves placement of implants, for example, a sling, a patch, or a mesh, under the bladder neck or the mid-urethra to provide a urethral support platform. Providing support to the mid-urethra maintains the urethra in the normal anatomical position and eliminates the symptoms of SUI.

The placement of a sling at the mid-urethra to treat SUI involves some procedural challenges. Such procedural challenges involve visualizing the mid-section of the sling during the implantation and/or identification of the twisting of the protective cover or sleeve of the sling during implantation. The midsection of the sling should typically be located where an anatomical site, such as a mid-urethra or bladder neck of the patient, needs to be supported. To visualize the mid-section of the sling, different types of colored markers or tabs are being used at a mid-section of the protective sleeve as a visual guide to allow the ready identification of the mid-section of the sling during the implantation. However, the designs of the market-available markers or tabs may still impede clear visualization of the sub-urethral portion of the sling and may not provide enough rigidity to the sleeve material at the central point and contribute to undesirable twisting of the sleeve of the sling during implantation.

Hence, there exists a need to develop a robust midline marker to mark the midsection of the sling which can be easily attached to the mid-section of the protective sleeve of the sling as a visual aid to allow ready identification of the mid-section of the sling during implantation and which provides rigidity to the sleeve material at the central point and inhibits twisting of the sleeve during the surgery.

### SUMMARY OF THE INVENTION

The present invention generally relates to systems and methods for treatment of stress urinary incontinence both male and female humans or animals.

The present invention may include a sleeve, a sling inserted within the sleeve, and a midline marker through which the sleeve may be routed.

The sleeve may comprise a first portion, a second portion, and an opening or gap between the first portion and the second portion.

The sleeve may comprise an opaque surface and a transparent surface. The opaque surface may be colored, and the transparent surface may be clear to provide contrast between the surfaces. The opening or gap may be positioned at the approximate mid-point of a length of the sleeve.

The sling may comprise an elongated member such as a rectangular, elongated strip comprised of a color that is different from the color of the opaque surface of the sleeve. One or both ends of the sling may be tapered.

The midline marker may aid in implantation of the sleeve and sling at a desired location with a patient's body by, e.g., inhibiting the sleeve from sliding off or otherwise being removed from the sling during placement of the sling.

The mid-section of the sleeve may be wrapped around the midline marker to form a channel through the center of the midline marker.

The midline marker may comprise a base and a pair of walls extending upwardly from the base.

The midline marker may comprise a first opening extending along the first wall and a second opening extending along the second wall. The first and second openings of the midline marker may be aligned.

The midline marker may comprise a third opening at or near a bottom of the midline marker.

The midline marker may be colored with a color different than both the color of the opaque surface of the sleeve and the color of the sling.

The present invention may include a pair of dilator tubes attached at the terminal ends of the sleeve. The dilator tubes may comprise a contrast color different than the colors of the opaque surface of the sleeve, the sling, and the midline marker for easy visualization of each component during the placement of the sling.

Each dilator tube may comprise a notch positioned at or near the proximal portion. The notch may be suitable for the entry of a needle of a delivery device for delivering the sleeve and sling assembly inside the body of a patient.

The delivery device for the implantation of the sling may include a needle attached to a handle. The needle may be sized and shaped to slidably fit within the lumens of the dilator tubes. The needle and the handle of the delivery device may be substantially in the same plane or in different planes, and the needle may include sections located in different planes. The needle may include a conical distal tip and may have a sharp or blunt end. The distal tip of the needle may be designed for suitably being placed inside the tapered closed of the dilator tube during the delivery of the implant system.

The present invention may provide a method for delivering a sling of a sling assembly at the mid-urethra of a patient to treat SUI. In the sling assembly, the transparent surface of the sleeve may pass through the slots or openings and wrap over the walls of the midline marker to form a channel over the central region of the midline marker and the sling may be positioned inside the pockets of the sleeve. During the procedure, the sling assembly may be delivered inside the body of a patient through the delivery device and adjusted at the correct treatment location by tracking the position of the midline marker which may be positioned at approximately mid-point of the sleeve of the sling. The medical operator may insert various cutting tools known in the art to cut the transparent surface of the sleeve through the channel formed over the center of the midline marker. This will enable the sleeve to be slid off from the sling and finally out of the body of the patient. Once the sleeve is separated, the medical operator may be able to grip the midline marker to remove, it leaving the sling positioned at the respective mid-urethra or bladder neck of the patient.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 illustrates a top view of a protective sleeve without a sling according to one embodiment.

FIG. 2 illustrates a top view of a sling according to one embodiment.

FIG. 3A illustrates a top view of a transparent side of the protective sleeve including the sling according to one embodiment.

FIG. 3B illustrates a top view of a substantially opaque side of the protective sleeve including the sling according to one embodiment.

FIG. 4A illustrates a top view of a transparent side of a colored protective sleeve including the sling according to one embodiment.

FIG. 4B illustrates a top view of a substantially opaque side of the colored protective sleeve including the sling according to one embodiment.

FIG. 5 illustrates a perspective view of a midline marker according to one embodiment.

FIG. 6 illustrates a side view of a midline marker according to one embodiment.

FIG. 7 illustrates a perspective front view of a midline marker according to one embodiment.

FIG. 8 illustrates a perspective view of an assembly of the midline marker of FIGS. 5-7 with the protective sleeve of FIG. 1 according to one embodiment.

FIG. 9 illustrates a perspective side view of the assembly of the midline marker and the protective sleeve as depicted in FIG. 8 according to one embodiment.

FIG. 10 illustrates a perspective top view of the assembly of the midline marker and the protective sleeve as depicted in FIG. 8 according to one embodiment.

FIG. 11 illustrates a perspective view of an assembly of the midline marker with the protective sleeve including the sling according to one embodiment.

FIG. 12 illustrates a top view of a dilator tube according to one embodiment.

FIG. 13 illustrates a top view of an assembly of the midline marker with the protective sleeve having the sling and the dilator tube according to one embodiment.

FIG. 14 illustrates a side view of the attachments of the protective sleeve with the dilator tube and the sling with the dilator tube.

FIG. 15 illustrates a top view of a sling delivery device according to one embodiment.

FIG. 16 illustrates a sling delivery system employing the full sling assembly of FIG. 13 and the sling delivery device of FIG. 15.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The invention provides a sling assembly 100 comprising an implantable sling 120, sized and shaped for providing support to the mid-urethra and maintaining the urethra in a normal anatomical position to eliminate the symptoms of SUI. The sling assembly 100 may also include a sleeve 110 for covering, at least partially, the implantable sling 120. The sleeve 110 may comprise a clear transparent surface 116 and a substantially opaque surface 118. The substantially opaque surface 118 may be colored as discussed in more detail below. The transparent surface 116 and substantially opaque surface 118 may be joined at their respective sides and ends to form one or more enclosed areas or pockets inside the sleeve 110. The implantable sling 120 may be positioned inside the one or more enclosed areas or pockets of the sleeve 110.

In some embodiments, the sling 120 may be free floating inside the sleeve 110. In such embodiments, the sling 120 may not be attached to the sleeve 110. However, in some embodiments, the sling 120 may not be free floating but instead may be temporarily/removably anchored or attached to the sleeve 110 in one or more locations.

In some embodiments, the top surface of the sleeve 110 may be transparent and the bottom surface of the sleeve 110 may be opaque. In some other embodiments, the bottom surface of the sleeve 110 may be transparent and the top surface of the sleeve 110 may be opaque. In some embodiments, the opaque surface 118 (e.g., the top surface or the bottom surface) may be colored. The opaque surface 118 of the sleeve 110 may have an opening or gap 115 at its mid-section. When the sling 120 is placed inside the sleeve 110, the mid-section of the sling 120 may be exposed outside of the sleeve 110 through this opening or gap 115.

In some embodiments, the transparent surface 116 of the sleeve 110 may form a looped portion at the mid-section by substantially wrapping around the body of a midline marker 130, such as when it passes through one or more apertures 132a, 132b, 132c positioned on the body of the midline marker 130. In some embodiments, the opaque surface 118 of the sleeve 110 may instead wrap around the body of the midline marker 130.

In some embodiments, the midline marker 130 may be easily attached to the mid-section of the transparent surface 116 of the sleeve 110 to aid in marking the midsection of the sling 120 and act as a visual aid during sling implantation. The midline marker 130 may further provide rigidity to the sleeve 110 at the central point and inhibit twisting of the sleeve 110 during the surgery.

The sling assembly 100 may also include first and second dilator tubes 140, 140a having longitudinally extending lumens and attached at the first end portion 114a and the second end portions 114b of the sleeve 110 respectively. In some embodiments, the dilator tubes 140, 140a may be attached to the ends of the sleeve 110, with the first dilator tube 140 being attached to the first end portion 114a of the sleeve 110 and the second dilator tube 140a being attached to the second end portion 114b of the sleeve 110. In other embodiments, the dilator tubes 140, 140a may also or alternatively be attached to the first end 122 and/or the second end 124 of the sling 120, with the first dilator tube 140 being attached to the first end 122 of the sling 120 and the second dilator tube 140b being attached to the second end 124 of the sling 120. In some embodiments, the first and/or second ends 122, 124 of the sling 120 may be tapered.

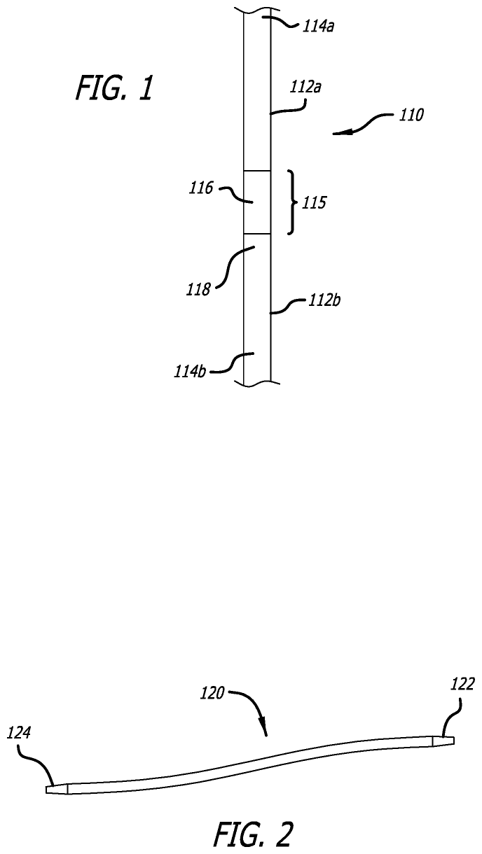

FIG. 1 illustrates a top view of an example embodiment of a sleeve 110. The sleeve 110 may include a transparent or semi-transparent surface 116 and an opaque or semi-opaque surface 118. As previously discussed, the transparent surface 116 may comprise a top surface of the sleeve 110 and the opaque surface 118 may comprise a bottom surface of the sleeve 110, or vice versa.

As shown in FIG. 1, the substantially opaque surface 118 of the sleeve 110 may not be continuous in an example embodiment. An opening or gap 115 may be located at the mid-portion of the substantially opaque surface 118 of the sleeve 110. The opening or gap 115 may comprise various types of openings, such as but not limited to a window or slot formed in one, but not both, of the surfaces 116, 118 of the sleeve 110.

Although the figures illustrate an example embodiment in which the opening or gap 115 is positioned at an approximate mid-point of the length of the sleeve 110, it should be appreciated that the position of the opening or gap 115 with respect to the end portions 114, 114a of the sleeve 110 may vary in different embodiments and should not be construed as limited by the example embodiments shown in the figures. In some embodiments such as shown in the figures, the opening or gap 115 may be positioned at the mid-point or approximate mid-point of the substantially opaque surface 118 of the sleeve 110. In some other embodiments, the opening or gap 115 may be positioned near the mid-portion of the substantially opaque surface 118 of the sleeve 110, but not exactly at the mid-portion, or even substantially offset from the mid-portion of the substantially opaque surface 118 of the sleeve 110.

In some embodiments, the sides and/or the ends of the transparent and opaque surfaces 116, 118 of the sleeve 110 may be joined together to form, for example, two pockets on both sides of the opening or gap 115 of the sleeve 110, with a first pocket located on a first side of the opening or gap 115 and a second pocket located on a second side of the opening or gap 115. The sleeve 110 may include a first end portion 114a and a second end portion 114b when the sides and the ends of the transparent and opaque surfaces 116, 118 of the sleeve 110 are joined together.

The sling assembly 100 may also include an implantable sling 120. The length of the sling 120 may vary in different embodiments to suit different anatomies and/or applications. In an example embodiment, the sling 120 may range from 405 millimeters to 420 millimeters in length after being cut. The sling 120 may comprise an elongated member such as a rectangular, elongated strip. However, it should be appreciated that the sling 120 may comprise other suitable shapes as known in the art in other example embodiments.

The sling 120 may comprise a uniform thickness over its entire length and/or width. Alternatively, the thickness of the sling 120 may vary at one or more locations along its length or width. In some embodiments, one or more portions of the sling 120 may be colored and one or more portions of the sling 120 may be clear. A wide range of colors may be utilized so long as the color may be contrasted with the color of the sleeve 110. A non-limiting example of the color of the sling 120 may be blue. Other colors that may be utilized include red, orange, yellow, green, blue, violet, or any other known color.

In an illustrative embodiment, FIG. 2 depicts a sling 120 which can be used for various medical purposes, such as but not limited to treatment of SUI. The sling 120 may be blue in color and may be fabricated from one or more yarns formed by biocompatible materials. In an example embodiment, sling 120 may have a uniform width throughout its length. In the embodiment shown in FIG. 2, it can be seen that the first and/or second ends 122, 124 of the sling 120 may be tapered. In some other embodiments, the sling 120 may have a uniform width over its entire length and/or width and thus may not include tapered ends.

The sling 120 may be comprised of a wide range of materials, and thus should not be construed as being limited to any particular material. Generally, the sling 120 may be flexible. In some embodiments, the sling 120 may be entirely made of various biocompatible materials known in the art. In some other embodiments, sling 120 may be fabricated from any of a number of flexible biocompatible materials. The materials of the sling 120 may be, for example, suitable to be absorbed by the patient's body. Alternatively, the material for the sling 120 may be derived from biodegradable materials. In an example embodiment, the sling 120 may be produced from a polypropylene homopolymer resin. The sling material may be fabricated from one or more yarns, and the yarns may be made from one or more materials. In some embodiments, the sling 120 may incorporate or be coated with one or more agents to provide a therapeutic effect, for example, to reduce discomfort, to reduce the chance of infection and/or to promote tissue growth.

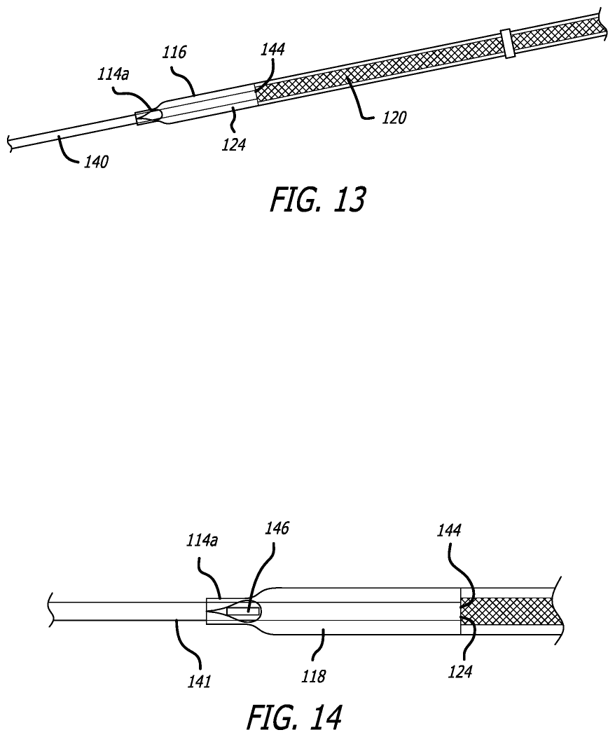

As previously mentioned, the implantable sling 120 may be positioned inside the pockets of the sleeve 110 formed by joining the sides and ends of the transparent and opaque surfaces 116, 118 of the sleeve 110. In some exemplary embodiments, such as shown in FIGS. 13 and 16, the length of the sling 120 may be shorter than the length of the sleeve 110. This feature may be helpful for the medical operator from inadvertently gripping the free ends of the sling 120 and tensioning the sling 120 during installation. In some exemplary embodiments, the sleeve 110 may be longer than the sling 120. In yet other exemplary embodiments, the sleeve 110 and sling 120 may be substantially the same length. It should thus be appreciated that the respective lengths of the sleeve 110 and sling 120 may vary in different embodiments and should not be construed as limited by the example embodiments shown in the figures.

In some embodiments, such as shown in FIGS. 13 and 16, the entire length of the sling 120 may not be connected to the entire length of the sleeve 110 such that the sling 120 may be moved within the sleeve 110. After sling placement, this feature may allow a medical operator to pull on the first end portion 114a and/or second end portion 114b of the sleeve 110, such as via the attached dilator tubes 140, 140a and/or any of the delivery devices to be used for placement, without the risk of stretching, curling or otherwise deforming the sling 120. However, in some embodiments, one or more portions of the sling 120 may be temporarily or removably attached to one or more portions of the sleeve 110.

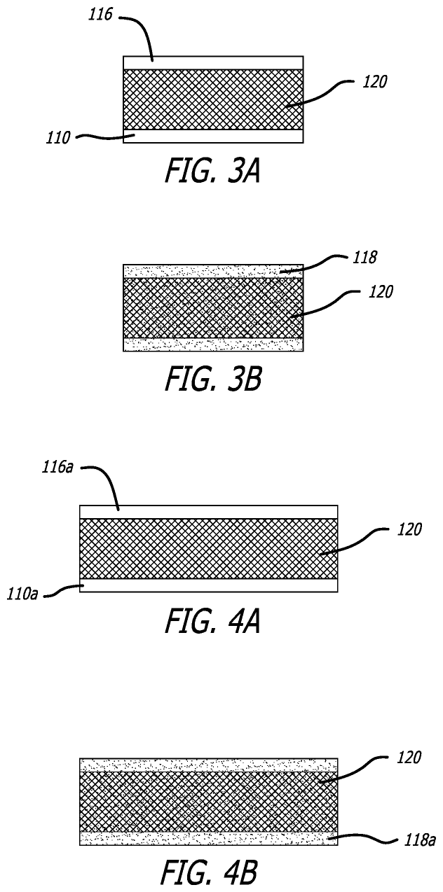

In an illustrative embodiment, FIG. 3A shows a cut out view of the portion of the sling 120 placed inside a portion of the sleeve 110. In this illustrative embodiment, the top of the sleeve 110 has a transparent surface 116. However, in another non-limiting embodiment, the bottom of the sleeve 110 may have a transparent surface and the top of the sleeve 110 may have a substantially opaque surface 118, as shown in FIG. 3B.

In some example embodiments, one surface of the sleeve 110 may be opaque and colored and the other surface of the sleeve 110 may be clear and fully or semi-transparent. Since the sleeve 110 is intended to designate the sub-urethral portion during the placement of the sling 120, the colored opaque surface 118 of the sleeve 110 may facilitate the installation by providing a contrast against the clear transparent surface 116 of the sleeve 110. In some embodiments, the contrast between the clear and transparent surface 116 and the colored opaque surface 118 of the sleeve 110 may help an operator to instantly determine if any twisting is present anywhere throughout the length of the sleeve 110.

The colored and/or opaque surface 118 of the sleeve 110 may also provide contrast between itself and the sling 120 and/or midline marker 130 to improve visualization of each component. Since one surface 116 of the sleeve 110 is clear and transparent, clear visualization of the sling 110 may not be hindered by the opaque and colored surface 118 of the sleeve 110. In some embodiments, the colored and opaque surface 118 of the sleeve 110 may also provide contrast between itself and first and second dilator tubes 140,140a to improve visualization of the entry point 146 of the dilator tube 140 for the entry of the delivery device 150.

In some exemplary embodiments, one surface of the sleeve 110 may be colored. By way of example, the opaque surface 118 of the sleeve 110 may be colored. Thus, the color of the opaque surface 118 of the sleeve 110 may be different than the color of the transparent surface 116 of the sleeve 110. The opaque surface 118 of the sleeve 110 may be colored or tinted and the transparent surface 116 of the sleeve 110 may be clear or white. In an example embodiment, the opaque surface 118 of the sleeve 110 may be colored as previously mentioned. A wide range of colors may be utilized for the opaque surface 118 of the sleeve 110, such as but not limited to pink. Other colors that may be utilized include red, orange, yellow, green blue, violet, and/or any other known color that contrasts with the color (or lack of color) of the transparent surface 116 of the sleeve and/or of the sling 120.

In an illustrative embodiment, FIG. 4A shows a cut out view of a portion of the sling 120 placed inside a portion of the sleeve 110a having both a colored and substantially opaque surface 118a and a clear and substantially transparent surface 116a. In the illustrated embodiment, the colored and substantially opaque surface 118a may comprise a bottom of the sleeve 110a and the clear and substantially transparent surface 116a may comprise the top of the sleeve 110a. However, in another non-limiting embodiment, the bottom of the sleeve 110a may have the clear and substantially transparent surface 116a and the top of the sleeve 110 may have the colored and substantially opaque surface 118a, as shown in FIG. 4B.

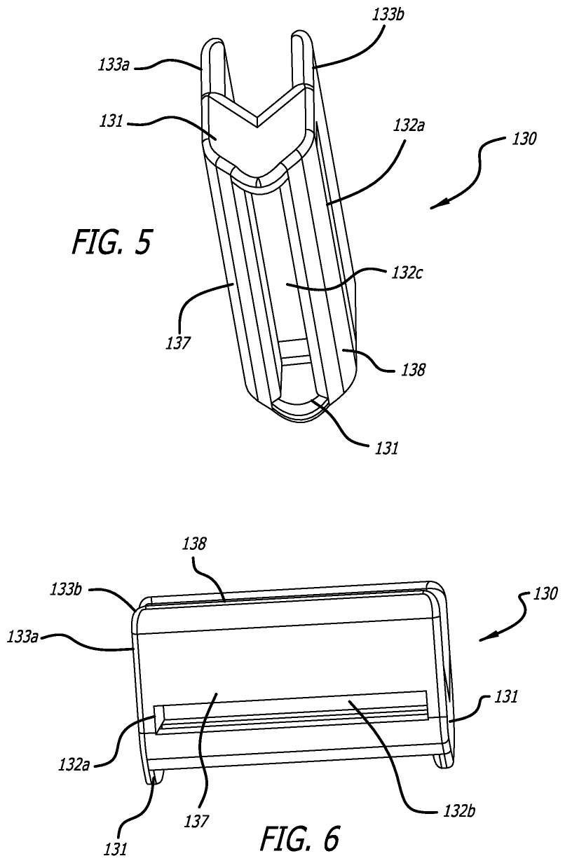

The sleeve 110a having a colored and opaque surface 118a and a clear and fully transparent surface 116a provide a contrast between the two surfaces 116a, 118a of the sleeve 110a to aid in identifying any twisting present anywhere throughout the length of the sleeve 110a. The colored and substantially opaque surface 118a of the sleeve 110a may also or alternatively provide a contrast between the sleeve 110a and the sling 120, and a midline marker 130 such as shown in FIGS. 5-7 to enable improved visualization of each of these components during the implantation of the sling 120.

As shown in FIGS. 5-11, the sling assembly 100 may also include a midline marker 130. The midline marker 130 is a sturdy component composed of bio-compatible materials. A non-limiting example of bio-compatible material for forming the midline marker may be polycarbonate polymer. In an example embodiment, the midline marker 130 may be composed of polycarbonate Makrolon 2458. In some other embodiments, a wide variety of other bio-compatible materials, such as polymers, metals, metal alloys, rigid plastics, stainless steel, and the like, may be used to form the midline marker 130. In some embodiments, the midline marker 130 may be easily attached to the mid-section of the transparent surface 116 of the sleeve 110 to help in marking the midsection of the sling 120 as a visual aid during sling implantation. Although the midline marker 130 is attached at the mid-point of the sleeve 110, in other embodiments, the midline marker 130 may be present near the mid-point of sleeve 110, but not exactly at the mid-point, or even substantially offset from the mid-point of the sleeve 110.

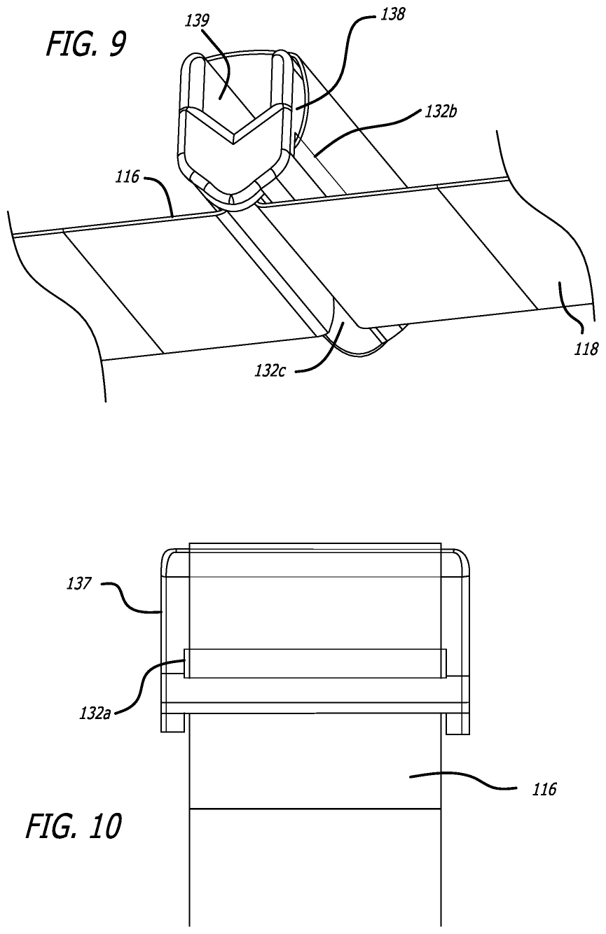

According to an example embodiment, the midline marker 130 may also inhibit the sleeve 110 from sliding off, or otherwise being removed from the sling 120 during sling placement. Preferably, the mid-section of the transparent surface of the sleeve 110 may be wrapped around the midline marker 130 to form a channel 139 through the center of the midline marker 130, such as shown in FIGS. 8-9. This channel is preferably large enough to enable a cutting instrument to enter the channel and execute a cut on the transparent surface 116 of the sleeve 110 during a procedure to enable the sleeve 110 to slide off the sling 120.

According to some embodiments, cutting the transparent surface 116 of the sleeve 110 over the center of the midline marker 130 enables the first portion 112a and the second portion 112b of the sleeve 110 to be separated from the sling 120 and finally out of the body of the patient by pulling the two dilator tubes 140 and 140a positioned on the two ends of the sling assembly 100. Once first portion 112a and the second portion 112b of the sleeve 110 are separated, the midline marker's sturdy material will enable the midline marker 130 to be gripped and removed deliberately either by the hand of the medical operator or by using an instrument.

FIGS. 5-7 depict a midline marker 130 according to one example embodiment of the present invention. The midline marker 130 may comprise a base 131 including a bottom, a first side 133a, and a second side 133b. The base 131 may be elongated between its front and rear sides such as shown in the figures. The bottom of the base 131 may be tapered to come to a rounded or curved point. Thus, as shown in the figures, the base 131 may form a pair of nose-shaped regions on the front and rear sides of the base 131.

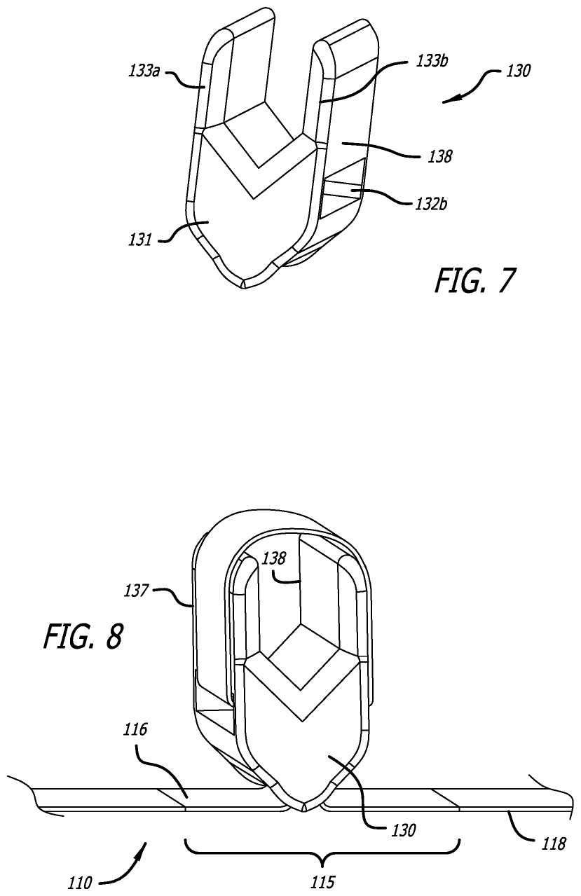

A pair of walls 137, 138 may extend upwardly from the base 131 such as best shown in FIGS. 6-7. More specifically, a first wall 137 may extend upwardly from the first side 133a of the base 131 and a second wall 138 may extend upwardly from the second side 133b of the base 131. The pair of walls 137, 138 may be parallel and spaced apart with respect to each other so as to define an opening or channel therebetween. The upper end of the base 131 may comprise a V-shaped groove such as shown in FIG. 8, the V-shaped groove defining the lower end of the opening or channel 139. Two pairs of struts may extend upwardly from the base 131 so as to support the walls 137, 138.

As can be best seen in FIG. 6, the midline marker may include a body comprising the base 131, the first wall 137, and the second wall 138. Thus, the base 131, the first wall 137, and the second wall 138 may be integrally formed (e.g., by being cast from the same mold) in some embodiments such as shown in the figures.

The first side 133a of the base 131 may include a first opening 132a extending between the front and rear sides of the midline marker 130. Similarly, the second side 133b of the base 131 may include a second opening 132b extending between the front and rear sides of the midline marker 130. The first and second openings 132a, 132b may be aligned such as shown in FIG. 6. In some embodiments, the first opening 132a may be positioned on the first wall 137 and the second opening 132b may be positioned on the second wall 138.

The shape, size, orientation, position, and type of openings 132a, 132b of the midline marker 130 may vary in different embodiments and thus should not be construed as limiting by the example embodiments shown in the figures. The openings 132a, 132b may comprise horizontal slots such as shown in the figures. Thus, the first opening 132a may comprise a first slot and the second opening 132b may comprise a second slot. Alternatively, the openings 132a, 132b may comprise various other types of openings or windows. While the figures illustrate an embodiment in which both openings 132a, 132b are substantially similar, in some embodiments the first opening 132a may be different than the second opening 132b.

In some embodiments, the first and second openings 132a, 132b may not extend to the proximal and distal ends of the first wall 137 and the second wall 138 of the midline marker 130. In some embodiments, the lengths of the first and second openings 132a, 132b may be smaller than the lengths of the first wall 137 and the second wall 138 of the midline marker 130.

It should be appreciated that the respective lengths of the first and second openings 132a, 132b may vary in different embodiments. The first and second openings 132a, 132b may be substantially the same size (e.g., length), or may be different sizes. Additionally, the ratio of the lengths of the respective first and second openings 132a, 132b to the overall size of the respective first and second walls 137, 138 or the base 131 may vary in different embodiments and should not be construed as limited by the exemplary embodiments shown in the figures. According to some embodiments, the smaller lengths of the first and second openings 132a, 132b than the lengths of the first wall 137 and the second wall 138 increase the sturdiness of the midline marker 130.

FIG. 5 further shows that the first wall 137 and the second wall 138 of the body of the midline marker 130 may terminate at the lower end of the base 131 so as to form a third opening 132c at the bottom of the base 131 of the midline marker 130. The third opening 132c may be oriented at a right angle with respect to the parallel first and second openings 132a, 132b. The shape, size, orientation, position, and type of the third opening 132c of the midline marker 130 may vary in different embodiments and thus should not be construed as limiting by the example embodiments shown in the figures. The third opening 132c may comprise a substantially horizontal slot such as shown in the figures. Alternatively, the third opening 132c may comprise various other types of openings or windows. The third opening 132c may be the size (e.g., length) as the first and second openings 132a, 132b.

In some embodiments, the midline marker 130 may be colored. A wide range of colors may be utilized for the midline marker 130. A non-limiting example of a color of the midline marker may be green. Other colors that may be utilized include red, orange, yellow, green, blue, violet, and any other known color. The color of the midline marker 130 may provide contrast against the clear and transparent surface 116 of the sleeve 110 and help in visualizing the channel 139 (as shown in FIG. 9) formed by the transparent surface 116 of the sleeve 110 over the midline marker 130. The channel 139 may be large enough to enable the cutting instrument to enter the channel and execute a cut on the transparent surface 116 of the sleeve 110 for separation of the sleeve 110 during a procedure.

In some embodiments, the midline marker may be easily attached to the mid-point of the transparent surface 116 of the sleeve 110 by passing the sleeve 110 through the first, second and third openings 132a, 132b, 132c. According to some embodiments, the sleeve 110 may enter and exit the midline marker 130 through the same opening, for example, the third opening 132c. Although, in some embodiments, the midline marker 130 may be attached at the mid-point of the transparent surface 116 of the sleeve 110, in some other embodiments, the midline marker 130 may be present near the mid-point of the transparent surface 116 of sleeve 110, but not exactly at the mid-point, or even substantially offset from the mid-point of the transparent surface 116 of the sleeve 110.

As can be seen in FIG. 8, the midline marker 130 may be attached to the midpoint of the transparent surface 116 of the sleeve 110 above the middle of the opening or gap 115 located near a mid-portion of the substantially opaque bottom surface 118 of the sleeve 110.

FIGS. 8-10 detail the mechanism of the attachment of the midline marker 130 with the transparent surface 116 of the sleeve 110. As can be seen in FIG. 9, first, the transparent surface 116 of the sleeve 110 may enter inside the midline marker 130 through the third opening 132c. Next, the transparent surface 116 of the sleeve 110 may exit from the opening 132b positioned on the second wall 138 and may wrap over the second wall 138 and the first wall 137 of the midline marker 130. Next, the transparent surface 116 of the sleeve 110 may enter inside the opening 132a (as best can be seen in FIG. 10) positioned on the first wall 137 and finally may exit from the third opening 132c (as shown in FIG. 9). In this way, the transparent surface 116 of the sleeve 110 may enter and exit the midline marker via the same opening 132c and wrap around the entirety of the midline marker 130.

FIG. 9 further shows that a channel 139 may be formed through the center of the midline marker 130 when the transparent surface 116 of the sleeve 110 is wrapped over the second wall 138 and the first wall 137 the midline marker 130. Thus, the channel may be defined between the upper end of the base 131, the inner edges of the walls 137, 138, and the sleeve 116 such as best shown in FIG. 9.

This channel 139 may be large enough to enable a cutting instrument to enter the channel 139 and execute a cut on the transparent surface 116 of the sleeve 110 during a procedure. The cut on the sleeve will enable the separation of the first portion 112a and the second portion 112b of the sleeve 110 as well as the separation of the midline marker 130 from the sleeve 110. Once the first portion 112a and the second portion 112b of the sleeve 110 are separated, the midline marker's 130 sturdy material and the first wall 137 and the second wall 138 on either side of the midline marker 130 will enable the midline marker 130 to be gripped and removed deliberately either by hand of the medical operator or using an instrument.

In some embodiments, the sides and the ends of the transparent and opaque surfaces 116,118 of the sleeve 110 are joined together to form, for example, two pockets on both sides of the opening or gap 115 of the sleeve 110. An implantable sling 120 may be positioned inside the pockets of the sleeve 110. When the sling 120 is placed inside the sleeve 110, the mid-portion of the sling 120 may be exposed outside of the sleeve 110 through this opening. In some exemplary embodiments (as best seen in FIGS. 13 and 16), the length of the sling 120 may be shorter than the length of the sleeve 110. This smaller length of the sling 120 helps the medical operator from inadvertently gripping the free ends of the sling 120 and tensioning the sling 120.

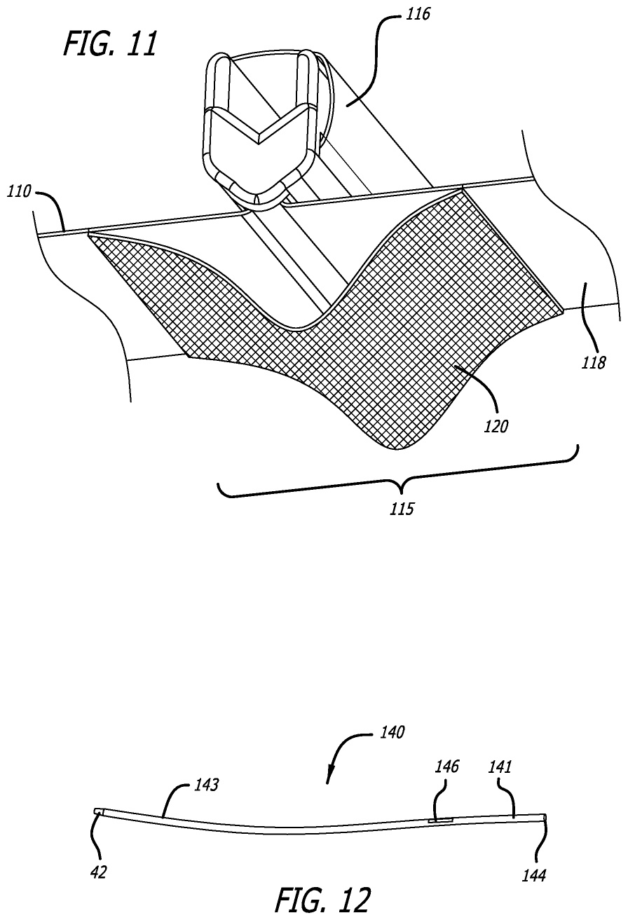

FIG. 11 shows an example embodiment in which the sling 120 may be placed inside the one or more pockets of the sleeve 110. In some embodiments, the midline marker 130 may be attached to the midpoint of the transparent surface 116 of the sleeve 110. FIG. 11 further shows that the opening or gap 115 located near a mid-portion of the opaque surface 118 of the sleeve 110 may expose a portion of the sling 120 where the entire width of the sling 120 is exposed when the sling 120 is placed inside the one or more pockets of the sleeve 110.

The sling assembly 100 may also include first and a second dilator tubes 140, 140a (as can be best seen in FIG. 16), possessing longitudinally extending through lumens and located at first and second ends of the sling assembly 100. In some embodiments, the dilator tubes 140,140a may be attached to first and second ends 114a, 114b of the sleeve 110. In other embodiments, the dilator tubes 140, 140a may additionally be attached to the terminal ends 122, 124 of the sling 120 which, as previously discussed, may be tapered in some example embodiments.

The longitudinally extending through lumen of the dilator tube 140 may have a uniform diameter or may include one or more locations of increased and/or decreased diameter. In some embodiments, the dilator tube 140 may terminate in one or more closed tapered ends which may have for example, a pointy end or a blunt end. Such a tapered end may help in percutaneous entry during a procedure.

In some embodiments, the dilator tube 140 may also be colored. A wide range of colors may be utilized. A non-limiting example of the color of the dilator tube 140 may be dark blue. Other colors that may be utilized include red, orange, yellow, green, blue, violet, and any known color. In some embodiments, the color of the dilator tube 140 may be distinctly different than the colors of the sleeve 110 and sling 120 to provide contrast and to help clearly visualize each element of the sling assembly 100 by the medical operator during a procedure.

In some embodiments, the dilator tube 140 may comprise a needle entry point 146 at its straight proximal portion 141. In some other example embodiments, the needle entry point 146 may be positioned towards the curved distal portion 143. The dilator tube 140 may comprise a wide range of materials and thus should not be construed as limited to any particular material. In some embodiments, the dilator tube 140 may be made of a flexible and bio-compatible polymer.

In an illustrative embodiment, FIG. 12 demonstrates a dilator tube 140 which may possess a longitudinally extending through-lumen having a uniform diameter. The internal and external diameter of the dilator tube 140 may vary in different embodiments to suit different anatomies and applications. In an example embodiment, the inner diameter of the dilator tube 140 may be between 2.75 millimeters and 315 millimeters. The size of the dilator tube 140 may vary depending on the size of the introducer. For example, with 2.7 millimeter introducers, the inner diameter of the dilator tube 140 may range from 2.75 millimeters to 2.85 millimeters and, with 3.0 millimeter introducers, the inner diameter of the dilator tube 140 may range from 3.05 millimeters to 3.15 millimeters.

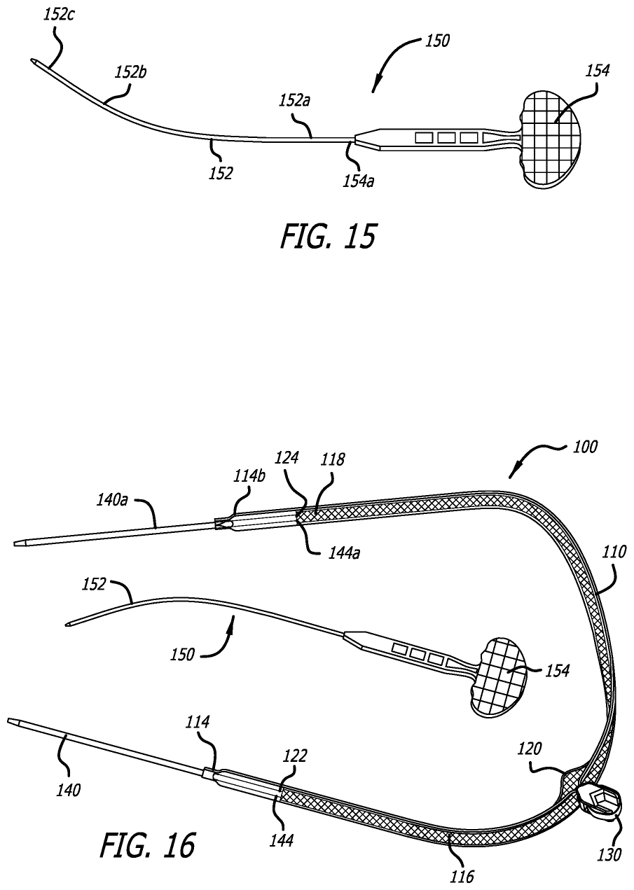

The dilator tube 140 may comprise a first proximal open end 144 and a second distal tapered end 142. The dilator tube 140 further may comprise a first straight proximal portion 141 and the first straight proximal portion 141 may extend distally to a curved distal portion 143. The first straight proximal portion 141 of the dilator tube 140 may comprise a notch 146 as an entry point of a needle 152 of a delivery device 150 as shown in FIG. 15. The colored dilator tube 140 may enable improved visualization of the needle entry point 146 as well as contrast against different colors of the sleeve 110 and the sling 120.

In some embodiments, the proximal portion 141 of the first dilator tube 140 may be attached to the first end portion 114a of the sleeve 110. The second dilator tube 140a may be similarly attached to the second end portion 114b of the sleeve 110. In some embodiments, the attachment of the dilator tube 140 to the first end portion 114a of the sleeve 110 and the attachment of the second dilator tube 140a to the second end portion 144b of the sleeve 110 may be made by any suitable mechanism, including, without limitation, heat bonding, gluing, stapling, stitching, shrink wrapping or the like. In some embodiments, the first tapered end 122 of the sling 120 may be attached to the first proximal open end 144 of the first dilator tube 140 and the second tapered end 124 of the sling 120 may be attached to the first proximal open end 144a of the second dilator tube 140a. The attachment between the sling 120 and the dilator tube 140 may be made by any suitable mechanism known in the art.

In some embodiments, the first end portion 114a of the sleeve 110 may be wrapped around the outer diameter of the proximal portion 141 of the first dilator tube 140 distally to the needle entry point 146 such that the entry point 146 for the needle 152 of the delivery device 150 is not hindered. Similarly, the second end portion 114b of the sleeve 110 may be wrapped around the outer diameter of the proximal portion 141a of the second dilator tube 140a distally to the needle entry point 146 such that the entry point of the needle 152 of the delivery device 150 is not hindered inside the second dilator tube 140a.

In an illustrative embodiment, FIG. 13 demonstrates that one of the tapered ends 124 of the sling 120 may be attached to the first open end 144 of the dilator tube 140. FIG. 13 further demonstrates that the transparent surface 116 of the first end 114a of the sleeve 110, as shown previously in FIG. 1, may be completely wrapped around and attached to the first straight proximal portion 141 of the dilator tube 140. The attachment between the first end portion 114a of the sleeve 110 and the first straight proximal portion 141 of the dilator tube 140 may be made by any suitable mechanism, as is known to those of ordinary skill in the art. In another illustrative embodiment, FIG. 14 shows that the opaque surface 118 of the first end 114a of the sleeve 110 may be partially wrapped around the first straight proximal portion 141 of the dilator tube 140 so that the entry point 146 positioned on the proximal portion 141 of the dilator tube 140 is not hindered for the entry of the needle 152 of the delivery device 150.

The sling assembly 100 may also benefit from a delivery device 150 for the implantation of the sling 120. According to some embodiments, the delivery device 150 may include a needle 152 attached to a handle 154 such as shown in FIG. 15. The needle 152 of the delivery device 150 may sized and shaped to slidably fit within the lumens of the first and second dilator tubes 140, 140a. According to one example embodiment, such a configuration may enable the first and second dilator tubes 140, 140a to rotate freely when the needle 152 is inserted through the entry point 146. Such rotation may reduce the twisting or other deformation of the sleeve 110, the sling 120, and the combination thereof during placement of the sling 120. The needle 152 and the handle 154 of the delivery device 150 may be substantially in the same plane or in different planes, and the needle 152 may include sections located in different planes. One or more parts or the needle 152 may assume a curved, angled, or any other suitable shape including substantially straight. The needle 152 may include a distal tip 152c, and in some non-limiting embodiments, the distal tip 152c may be conical in shape and may have a sharp or blunt end. The distal tip 152c of the needle may be designed for suitably being placed inside the tapered closed end 142 of the dilator tube 140.

FIG. 15 illustrates an example embodiment of a delivery device 150. The delivery device 150 may comprise a handle 154 and a needle 152. The shaft of the needle 152 may be permanently attached to the handle 154 at a proximal end 154a and may contain a first substantially straight portion 152a. The first substantially straight portion 152a of the needle 152 may extend distally to a curved portion 152b and terminate at a distal tip 152c. The distal tip 152c may be conical in shape and may have a sharp end. The sharp end of the tip 152c may be designed for percutaneous punctuation and/or advancement through tissue of a patient. The shaft of the needle 152 is sized and shaped to fit within the lumen of the dilator tube 140 and the needle enters inside the dilator tube 140 through the notch 146, as shown in FIGS. 12 and 14.

The shaft of the needle 152 may be formed from a rigid material such as, for example, a metal, metal alloy, or polymer. A variety of components may be used to form the shaft of the needle 152. In one embodiment, the needle may be made of stainless steel. In some other non-limiting examples, the needle may be made of other rigid or semi-rigid bio-compatible compounds known in the art. In some example embodiments, the one or more surfaces of the handle 154 of the delivery device 150 may be textured so as to provide increased traction relative to a gloved hand of a medical operator.

FIG. 16 shows a perspective view of a sling assembly 100 including the delivery device 150. The sling assembly 100 may include a sling 120 inserted to extend through a sleeve 110 having a transparent surface 116 and an opaque surface 118, with the transparent surface 116 being clear and the opaque surface 118 being colored. A midline marker 130 may be attached at the midpoint of the transparent surface 116 of the sleeve 110. FIG. 16 further shows that the first tapered end 122 of the sling 120 may be attached to the open end 144 of the first dilator tube 140 and the second tapered end 124 of the sling 120 may be attached to the first open end 144a of the second dilator tube 140a. FIG. 16 also shows that the transparent surface 116 of the first end portion 114a of the sleeve 110 is completely wrapped around and attached to the first dilator tube 140 and the substantially opaque and colored surface 118 of the second end portion 114b of the sleeve 110 is partially wrapped around the second dilator tube 140a leaving the entry point 146 unhindered for inserting the needle 152 of the delivery device 150.

According to some embodiments, the invention may provide a method for treating stress urinary incontinence by implanting a surgical sling 120 into the body of a patient. In one such procedure, the sling assembly 100 may be introduced near the mid-urethra by the delivery device 150 through an incision in the vaginal wall. In such a procedure, it may be beneficial to visualize the mid-section of the sling 120 and/or identify any twisting of the protective cover or sleeve 110 of the sling 120 during implantation.

In the present invention, the presence of the colored midline marker 130 at or near the mid-point of the transparent surface 116 of the sleeve 110 may enable a medical operator to clearly visualize the mid-section of the sling 120 during the implantation. Moreover, the substantially opaque and colored surface 118 of the sleeve 110 may provide contrast between the clear, transparent surface 116 and the colored opaque surface 118 of the sleeve 110 to instantly determine if any twisting is present anywhere throughout the length of the sleeve 110. Once the sling 120 of the sling assembly 150 is adjusted at the correct treatment location by tracking the position of the midline marker 130, the medical operator may cut the transparent surface 116 of the sleeve 110 through the channel 139 formed over the center of the midline marker 130 by various cutting tools known in the art. This will enable the first portion 112a and the second portion 112b of the sleeve 110 to be slid off from the sling 120 and finally out of the body of the patient by pulling the two dilator tubes 140 and 140a on the two ends of the sling assembly 100 by the delivery device 150. Once the sleeve 110 is separated, the medical operator may be able to grip the sturdy material of the midline marker 130 to remove it either by the hand or by using an instrument leaving the sling 120 positioned at the respective mid-urethra or bladder neck of the patient.

The midline marker 130 may be designed to be very small so that it will not impede vision at the vaginal incision.

The midline marker 130 may enable the sleeve 110 to wrap around to enable the cut point at the sleeve to be separated from the sling 120, ensuring the sling 120 will not be damaged during removal of the midline marker 130.

The midline marker 130 may provide rigidity to the sleeve material 110 at the central point, inhibiting twisting of the sleeve 110 during operation.

The midline marker 130 may be designed to not impede sub-urethral positioning of the sling 120 including the entirety of the mesh included in the sling 120.

The midline marker cutting channel 139 may be chamfered to guide the cutting instrument across the channel as the cut is being executed.

The midline marker 130 may additionally be gripped during the tensioning of the sling 120.

The midline marker 130 may provide a midpoint to determine the position of the sling 120 compared to the urethra.

The midline marker 130 may provide structural support and rigidity at the center of the sleeve 110 to prevent twisting.

The colored sleeve 110a may provide contrast between itself and the dilator tubes 140, 140a to enable superior visualization of the needle entry point 139 in the tubes.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope there.

What is claimed is:

1. A midline marker for a sling implant, comprising:
a base comprising a bottom, a first side, and a second side;
a first wall extending upwardly from the first side of the base; and
a second wall extending upwardly from the second side of the base, the second wall being parallel to the first wall so as to define channel between the first wall and the second wall;
wherein the first side of the base includes a first opening;
wherein the second side of the base includes a second opening; and,
wherein the bottom of the base includes a third opening.

2. The midline marker of claim 1, wherein the first opening of the first side is aligned with the second opening of the second side.

3. The midline marker of claim 1, wherein a length of the first opening of the first side is equal to a length of the second opening of the second side.

4. The midline marker of claim 3, wherein a length of the third opening is equal to the length of the first opening and the length of the second opening.

5. The midline marker of claim 1, wherein the bottom of the base is tapered.

6. The midline marker of claim 1, wherein an upper end of the base comprises a V-shaped groove.

7. The midline marker of claim 1, wherein the third opening is positioned between the first opening and the second opening.

8. A sleeve for a sling implant, comprising:
a first surface; and
a second surface having an opening therein, the second surface comprising a different color than the first surface; and,
wherein the first surface and the second surface are joined at their respective edges to form pockets on both sides of the opening for receiving the sling implant.

9. The sleeve of claim 8, wherein the first surface of the sleeve is substantially transparent.

10. The sleeve of claim 9, wherein the second surface of the sleeve is substantially opaque.

11. The sleeve of claim 8, wherein the first surface is clear and wherein the second surface is colored.

12. The sleeve of claim 8, wherein the implantable sling is at least partially exposed through the opening of the sleeve.

13. The sleeve of claim 8, wherein a first dilator tube is attached to a first end of the sleeve.

14. The sleeve of claim 13, wherein a second dilator tube is attached to a second end of the sleeve.

15. The sleeve of claim 8, wherein the opening is positioned at a mid-section of the second surface.

16. A method of installing a sleeve on a midline marker comprising:
inserting a first end of the sleeve through a bottom slot and a first side slot of the midline marker;
passing the first end of the sleeve over a first wall and a second wall of the midline marker; and
inserting the first end of the sleeve through a second side slot and the bottom slot of the midline marker; and,
wherein a channel is defined between the sleeve and the midline marker.

17. The method of claim 16, further comprising the step of inserting a cutting tool inside the channel and cutting the sleeve.

18. The method of claim 16, further comprising the step of inserting a sling implant within the sleeve.

19. The method of claim 16, wherein the sleeve comprises a clear transparent surface and a colored opaque surface.

20. The method of claim 19, wherein the midline marker is a different color than the colored opaque surface of the sleeve.
